(19) United States
(12) Patent Application Publication
(10) Pub. No.: US 2006/0058525 A1
(43) Pub. Date: Mar. 16, 2006

(54) Synthesis of 2,4-pyrimidinediamine compounds

(76) Inventors: Rajinder Singh, Belmont, CA (US); Ankush Argade, Foster City, CA (US)

Correspondence Address: DECHERT LLP, P.O. BOX 10004, PALO ALTO, CA, 94303, US

(21) Appl. No.: 11/216,532

(22) Filed: Aug. 30, 2005

Related U.S. Application Data
(60) Provisional application No. 60/606,380, filed on Sep. 1, 2004.

(51) Int. Cl. A61K 31/506; C07D 239/48

(52) U.S. Cl. 544/297; 544/326; 544/328

(57) ABSTRACT

The present invention provides an economical and efficient method to prepare various substituted 2,4-pyrimidinediamine compounds in large scale quantities.

### CROSS-REFERENCE TO RELATED APPLICATION

This application claims benefit under 35 U.S.C. § 119(e) to application Ser. No. 60/606,380 filed Sep. 1, 2004, entitled “SYNTHESIS OF 2,4-PYRIMIDINEDIAMINE COMPOUNDS,” the contents of which are incorporated herein by reference.

### FIELD OF THE INVENTION

The present invention relates generally to improved synthetic methods of making 2,4-pyrimidinediamine compounds.

### BACKGROUND OF THE INVENTION

2,4-Pyrimidinediamine compounds have been found to be potent inhibitors of degranulation of immune cells, such as mast, basophil, neutrophil and/or eosinophil cells. As such, 2,4-pyrimidinediamine compounds can provide methods of regulating, and in particular inhibiting, degranulation of such cells. Treatment generally involves contacting a cell that degranulates with an amount of a 2,4-pyrimidinediamine compound or prodrug thereof, or an acceptable salt, hydrate, solvate, N-oxide and/or composition thereof, effective to regulate or inhibit degranulation of the cell. Examples include anaphylactoid reactions, hay fever, allergic conjunctivitis, allergic rhinitis, allergic asthma, atopic dermatitis, eczema, urticaria, mucosal disorders, tissue disorders and certain gastrointestinal disorders.

Preparation of 2,4-pyrimidinediamines has been accomplished generally in multistep procedures. For example, a representative multi-step procedure involves condensation of a guanidine with an enaminone. Appropriate guanidines must be prepared prior to the condensation reaction by reaction between a suitable aniline with cyanamide. Likewise, enaminones must also be prepared by a reaction of an acetyl compound and an acetal. As a consequence, this synthesis suffers from the drawback that the acetyl and acetal compounds often need to be prepared prior to use as well.

It is also appreciated by process development specialists that many processes, procedures, and/or reactions are not amenable to being carried out on a large scale as is done in a pilot plant or a manufacturing facility. Some examples of situations where scale-up can be problematic may involve the use of hazardous or toxic reagents and/or solvents; highly exothermic reactions; high pressure or high vacuum processes, such as those required for certain high pressure reactions or high vacuum distillations; chromatographic separation and/or purification. Also troublesome are processes exhibiting reduced yield on scale-up and the like. A more recent consideration for large scale operations is the limitations that have been set on certain emissions as well as the disposal of waste products from chemical processing. Processes involving these aspects incur higher levels of cost in production.

There is a need for an improved method to prepare 2,4-pyrimidinediamine compounds that overcomes one or more drawbacks of current syntheses.

### BRIEF SUMMARY OF THE INVENTION

The present invention pertains to methods of synthesizing 2,4-pyrimidinediamine compounds according to structural formula (I): 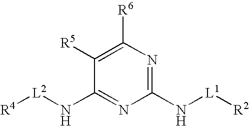
and salts, hydrates, solvates, N-oxides and prodrugs thereof, wherein:
L¹ and L² are each, independently of one another, selected from the group consisting of a direct bond and a linker;
R² is selected from the group consisting of (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;
R⁵ is selected from the group consisting of R⁶, (C1-C6) alkyl optionally substituted with one or more of the same or different R⁸ groups, (C1-C4) alkanyl optionally substituted with one or more of the same or different R⁸ groups, (C2-C4) alkenyl optionally substituted with one or more of the same or different R⁸ groups and (C2-C4) alkynyl optionally substituted with one or more of the same or different R⁸ groups;
each R⁶ is independently selected from the group consisting of hydrogen, an electronegative group, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CF₃, —CH₂CF₃, —CF₂CF₃, —CN, —NC, —OCN, —SCN, —NO, —NO₂, —N₃, —S(O)R^{d}, —S(O)₂R^{d}, —S(O)₂OR^{d}, S(O)NR^{c}R^{c}; S(O)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙNR^{c}R^{c} and —[NHC(NH)]ₙNR^{c}R^{c}, (C5-C10) aryl optionally substituted with one or more of the same or different R⁸ groups, phenyl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups and 6-16 membered heteroarylalkyl optionally substituted with one or more of the same or different R⁸ groups;
R⁸ is selected from the group consisting of R^{a}, R^{b}, R^{a} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘR^{b}, —S—(CH₂)ₘ—R^{b}, —O—CHR^{a}R^{b}, —O—CR^{a}(R^{b})₂, —O(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
   each R^{a} is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
   each R^{b} is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
   each R^{c} is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
   each R^{d} is independently a protecting group or R^{a};
   each m is independently an integer from 1 to 3; and
   each n is independently an integer from 0 to 3.

In one embodiment, the present invention pertains to methods of synthesizing 2,4-pyrimidinediamine compounds according to structural formula (Ia): 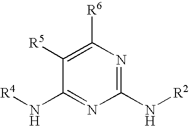
including salts, hydrates, solvates and N-oxides thereof, wherein R², R⁴, R⁵ and R⁶ are as previously defined for structural formula (I).

The synthesis includes treating a compound according to structural formula (II) 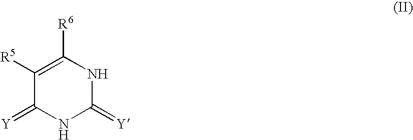
in step (a) with a phosphorous oxyhalide in an N,N-dialkylaniline at an elevated temperature wherein Y and Y′ are each, independently of one another, selected from the group consisting of O and S, to form a compound according to structural formula (III) 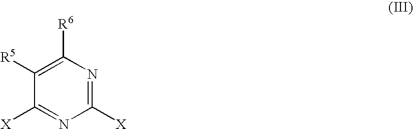
wherein each X is a halogen.

In step (b), compound (III) is treated in a solvent at an elevated temperature with an equivalent of a compound according to structural formula (IV)
R⁴-L²-NH₂ (IV)
thereby forming a compound according to structural formula (V)

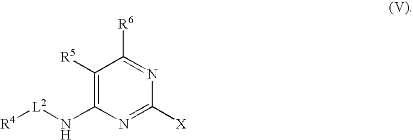

In step (c), compound (V) is treated with an equivalent of a compound according to the structural formula (VI)
R²-L¹-NH₂ (VI)
in a solvent at an elevated temperature to form compound (I), wherein R², R⁴, R⁵, R⁶, L¹ and L² are as defined above. In certain embodiments, L¹ and L² are direct bonds. In another embodiment R⁵ is a fluoride and R⁶ is a hydrogen. In still yet another embodiment, L¹ and L² are direct bonds, R⁵ is a fluoride and R⁶ is a hydrogen.

The reaction product of the present synthesis is a hydrochloride salt. The hydrochloride salt can be converted into other salts by exchange methods or the salt can be removed from the product by treatment with a basic solution.

The present invention provides the advantage that 2,4-pyrimidinediamine compounds are formed as slats, which can be washed with solvent to remove organic impurities. This simple purification step is efficient and reduces cost of purification. Additionally, the solvent can be recovered and recycled. The present invention avoids costly and inefficient purification procedure such as chromatography.

Typically, the phosphorous oxyhalide in step (a) is phosphorous oxychloride and the N,N-dialkylaniline is generally N,N-dimethylaniline. The reaction temperature of step (a) is generally performed in a range of between about 80° C. to about 85° C. The reaction temperature of step (b) and/or (c) is generally performed in a range of between about 60° C. and about 110° C.

In one specific embodiment, the present invention pertains to methods of synthesizing the 2,4-pyrimidinediamine compound according to structural formula: 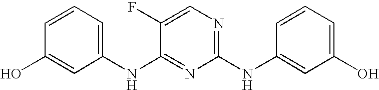
including salts, hydrates, solvates and N-oxides thereof.

### BRIEF DESCRIPTION OF THE DRAWINGS

FIG. 1 is a flow chart depicting a general synthetic method of the invention useful to prepare 2,4-pyrimidinediamine compounds;

FIG. 2 provides a cartoon illustrating the FcεR1 signal transduction cascade leading to degranulation of mast and/or basophil cells; and

FIG. 3 provides a cartoon illustrating the putative points of action of compounds that selectively inhibit upstream FcεRI-mediated degranulation and compounds that inhibit both FcεRI-mediated and ionomycin-induced degranulation.

### DETAILED DESCRIPTION OF THE INVENTION

As used herein, the following terms are intended to have the following meanings:

“Alkyl” by itself or as part of another substituent refers to a saturated or unsaturated branched, straight chain or cyclic monovalent hydrocarbon radical having the stated number of carbon atoms (i.e., C1 C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan 1 yl, propan 2 yl, cyclopropan 1 yl, prop 1 en 1 yl, prop 1 en 2 yl, prop 2 en 1 yl, cycloprop 1 en 1 yl; cycloprop 2 en 1 yl, prop 1 yn 1 yl, prop 2 yn 1 yl, etc.; butyls such as butan 1 yl, butan 2 yl, 2 methyl propan 1 yl, 2 methyl propan 2 yl, cyclobutan 1 yl, but 1 en 1 yl, but 1 en 2 yl, 2 methyl prop 1 en 1 yl, but 2 en 1 yl, but 2 en 2 yl, buta 1,3 dien 1 yl, buta 1,3 dien 2 yl, cyclobut 1 en 1 yl, cyclobut 1 en 3 yl, cyclobuta 1,3 dien 1 yl, but 1 yn 1 yl, but 1 yn 3 yl, but 3 yn 1 yl, etc.; and the like. Where specific levels of saturation are intended, the nomenclature “alkanyl,” “alkenyl” and/or “alkynyl” is used, as defined below. In preferred embodiments, the alkyl groups are (C1 C6) alkyl.

“Alkanyl” by itself or as part of another substituent refers to a saturated branched, straight chain or cyclic alkyl derived by the removal of one hydrogen atom from a single carbon atom of a parent alkane. Typical alkanyl groups include, but are not limited to, methanyl; ethanyl; propanyls such as propan 1 yl, propan 2 yl (isopropyl), cyclopropan 1 yl, etc.; butanyls such as butan 1 yl, butan 2 yl (sec-butyl), 2 methyl propan 1 yl (isobutyl), 2 methyl propan 2 yl (t butyl), cyclobutan 1 yl, etc.; and the like. In preferred embodiments, the alkanyl groups are (C1 C6) alkanyl.

“Alkenyl” by itself or as part of another substituent refers to an unsaturated branched, straight chain or cyclic alkyl having at least one carbon carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop 1 en 1 yl, prop 1 en 2 yl, prop 2 en 1 yl, prop 2 en 2 yl, cycloprop 1 en 1 yl; cycloprop 2 en 1 yl; butenyls such as but 1 en 1 yl, but 1 en 2 yl, 2 methyl prop 1 en 1 yl, but 2 en 1 yl, but 2 en 2 yl, buta 1,3 dien 1 yl, buta 1,3 dien 2 yl, cyclobut 1 en 1 yl, cyclobut 1 en 3 yl, cyclobuta 1,3 dien 1 yl, etc.; and the like. In preferred embodiments, the alkenyl group is (C2 C6) alkenyl.

“Alkynyl” by itself or as part of another substituent refers to an unsaturated branched, straight chain or cyclic alkyl having at least one carbon carbon triple bond derived by the removal of one hydrogen atom from a single carbon atom of a parent alkyne. Typical alkynyl groups include, but are not limited to, ethynyl; propynyls such as prop 1 yn 1 yl, prop 2 yn 1 yl, etc.; butynyls such as but 1 yn 1 yl, but 1 yn 3 yl, but 3 yn 1 yl, etc.; and the like. In preferred embodiments, the alkynyl group is (C2 C6) alkynyl.

“Alkyldiyl” by itself or as part of another substituent refers to a saturated or unsaturated, branched, straight chain or cyclic divalent hydrocarbon group having the stated number of carbon atoms (i.e., C1 C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan 1,1 diyl, ethan 1,2 diyl, ethen 1,1 diyl, ethen 1,2 diyl; propyldiyls such as propan 1,1 diyl, propan 1,2 diyl, propan 2,2 diyl, propan 1,3 diyl, cyclopropan 1,1 diyl, cyclopropan 1,2 diyl, prop 1 en 1,1 diyl, prop 1 en 1,2 diyl, prop 2 en 1,2 diyl, prop 1 en 1,3 diyl, cycloprop 1 en 1,2 diyl, cycloprop 2 en 1,2 diyl, cycloprop 2 en 1,1 diyl, prop 1 yn 1,3 diyl, etc.; butyldiyls such as, butan 1,1 diyl, butan 1,2 diyl, butan 1,3 diyl, butan 1,4 diyl, butan 2,2 diyl, 2 methyl propan 1,1 diyl, 2 methyl propan 1,2 diyl, cyclobutan 1,1 diyl; cyclobutan 1,2 diyl, cyclobutan 1,3 diyl, but 1 en 1,1 diyl, but 1 en 1,2 diyl, but 1 en 1,3 diyl, but 1 en 1,4 diyl, 2 methyl prop 1 en 1,1 diyl, 2 methanylidene propan 1,1 diyl, buta 1,3 dien 1,1 diyl, buta 1,3 dien 1,2 diyl, buta 1,3 dien 1,3 diyl, buta 1,3 dien 1,4 diyl, cyclobut 1 en 1,2 diyl, cyclobut 1 en 1,3 diyl, cyclobut 2 en 1,2 diyl, cyclobuta 1,3 dien 1,2 diyl, cyclobuta 1,3 dien 1,3 diyl, but 1 yn 1,3 diyl, but 1 yn 1,4 diyl, buta 1,3 diyn 1,4 diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group is (C1 C6) alkyldiyl. Also preferred are saturated acyclic alkanyldiyl groups in which the radical centers are at the terminal carbons, e.g., methandiyl (methano); ethan 1,2 diyl (ethano); propan 1,3 diyl (propano); butan 1,4 diyl (butano); and the like (also referred to as alkylenos, defined infra).

“Alkyleno” by itself or as part of another substituent refers to a straight chain saturated or unsaturated alkyldiyl group having two terminal monovalent radical centers derived by the removal of one hydrogen atom from each of the two terminal carbon atoms of straight chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1 C6) or (C1 C3) alkyleno. Also preferred are straight chain saturated alkano groups, e.g., methano, ethano, propano, butano, and the like.

“Heteroalkyl,” Heteroalkanyl,” Heteroalkenyl,” Heteroalkynyl,” Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, O, S, —S—O—, NR′, —PH—, S(O), S(O)₂, S(O)NR′, S(O)2NR′, and the like, including combinations thereof, where each R′ is independently hydrogen or (C1 C6) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, O, S, —S—O—, NR′, —PH—, S(O), S(O)₂, S(O)NR′, S(O)2NR′, and the like, including combinations thereof, where each R′ is independently hydrogen or (C1 C6) alkyl.

“Parent Aromatic Ring System” refers to an unsaturated cyclic or polycyclic ring system having a conjugated p electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s indacene, indane, indene, naphthalene, octacene, octaphene, octalene, ovalene, penta 2,4 diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as indacene, s indacene, indane, indene, naphthalene, octacene, octaphene, octalene, ovalene, penta 2,4 diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C5 C15) aryl, with (C5 C10) being even more preferred. Particularly preferred aryls are cyclopentadienyl, phenyl and naphthyl.

“Arylaryl” by itself or as part of another substituent refers to a monovalent hydrocarbon group derived by the removal of one hydrogen atom from a single carbon atom of a ring system in which two or more identical or non identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl naphthyl, binaphthyl, biphenyl naphthyl, and the like. Where the number of carbon atoms in an arylaryl group is specified, the numbers refer to the carbon atoms comprising each parent aromatic ring. For example, (C5 C15) arylaryl is an arylaryl group in which each aromatic ring comprises from 5 to 15 carbons, e.g., biphenyl, triphenyl, binaphthyl, phenylnaphthyl, etc. Preferably, each parent aromatic ring system of an arylaryl group is independently a (C5 C15) aromatic, more preferably a (C5 C10) aromatic. Also preferred are arylaryl groups in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. Preferably, the aromatic ring systems are (C5 C15) aromatic rings, more preferably (C5 C10) aromatic rings. A particularly preferred biaryl group is biphenyl.

“Arylalkyl” by itself or as part of another substituent refers to an acyclic alkyl group in which one of the hydrogen atoms bonded to a carbon atom, typically a terminal or sp3 carbon atom, is replaced with an aryl group. Typical arylalkyl groups include, but are not limited to, benzyl, 2 phenylethan 1 yl, 2 phenylethen 1 yl, naphthylmethyl, 2 naphthylethan 1 yl, 2 naphthylethen 1 yl, naphthobenzyl, 2 naphthophenylethan 1 yl and the like. Where specific alkyl moieties are intended, the nomenclature arylalkanyl, arylakenyl and/or arylalkynyl is used. In preferred embodiments, the arylalkyl group is (C6 C21) arylalkyl, e.g., the alkanyl, alkenyl or alkynyl moiety of the arylalkyl group is (C1 C6) and the aryl moiety is (C5 C15). In particularly preferred embodiments the arylalkyl group is (C6 C13), e.g., the alkanyl, alkenyl or alkynyl moiety of the arylalkyl group is (C1 C3) and the aryl moiety is (C5 C10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)2, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Specifically excluded from the definition of “parent heteroaromatic ring system” are benzene rings fused to cyclic polyalkylene glycols such as cyclic polyethylene glycols. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, b carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5 14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, b carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5 14 membered heteroaryl, with 5 10 membered heteroaryl being particularly preferred.

“Heteroaryl Heteroaryl” by itself or as part of another substituent refers to a monovalent heteroaromatic group derived by the removal of one hydrogen atom from a single atom of a ring system in which two or more identical or non identical parent heteroaromatic ring systems are joined directly together by a single bond, where the number of such direct ring junctions is one less than the number of parent heteroaromatic ring systems involved. Typical heteroaryl heteroaryl groups include, but are not limited to, bipyridyl, tripyridyl, pyridylpurinyl, bipurinyl, etc. Where the number of atoms is specified, the numbers refer to the number of atoms comprising each parent heteroaromatic ring systems. For example, 5 15 membered heteroaryl heteroaryl is a heteroaryl heteroaryl group in which each parent heteroaromatic ring system comprises from 5 to 15 atoms, e.g., bipyridyl, tripuridyl, etc. Preferably, each parent heteroaromatic ring system is independently a 5 15 membered heteroaromatic, more preferably a 5 10 membered heteroaromatic. Also preferred are heteroaryl heteroaryl groups in which all of the parent heteroaromatic ring systems are identical.

“Biheteroaryl” by itself or as part of another substituent refers to a heteroaryl heteroaryl group having two identical parent heteroaromatic ring systems joined directly together by a single bond. Typical biheteroaryl groups include, but are not limited to, bipyridyl, bipurinyl, biquinolinyl, and the like. Preferably, the heteroaromatic ring systems are 5 15 membered heteroaromatic rings, more preferably 5 10 membered heteroaromatic rings.

“Heteroarylalkyl” by itself or as part of another substituent refers to an acyclic alkyl group in which one of the hydrogen atoms bonded to a carbon atom, typically a terminal or sp3 carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6 21 membered heteroarylalkyl, e.g., the alkanyl, alkenyl or alkynyl moiety of the heteroarylalkyl is (C1 C6) alkyl and the heteroaryl moiety is a 5 15 membered heteroaryl. In particularly preferred embodiments, the heteroarylalkyl is a 6 13 membered heteroarylalkyl, e.g., the alkanyl, alkenyl or alkynyl moiety is (C1-C3) alkyl and the heteroaryl moiety is a 5 10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR′″, where R′″ is a haloalkyl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3rd Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated or alkylated such as benzyl and trityl ethers, as well as alkyl ethers, tetrahydropyranyl ethers, trialkylsilyl ethers (e.g., TMS or TIPPS groups) and allyl ethers.

“Prodrug” refers to a derivative of an active 2,4-pyrimidinediamine compound (drug) that requires a transformation under the conditions of use, such as within the body, to release the active 2,4-pyrimidinediamine drug. Prodrugs are frequently, but not necessarily, pharmacologically inactive until converted into the active drug. Prodrugs are typically obtained by masking a functional group in the 2,4-pyrimidinediamine drug believed to be in part required for activity with a progroup (defined below) to form a promoiety which undergoes a transformation, such as cleavage, under the specified conditions of use to release the functional group, and hence the active 2,4-pyrimidinediamine drug. The cleavage of the promoiety may proceed spontaneously, such as by way of a hydrolysis reaction, or it may be catalyzed or induced by another agent, such as by an enzyme, by light, by acid or base, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

A wide variety of progroups, as well as the resultant promoieties, suitable for masking functional groups in the active 2,4-pyrimidinediamines compounds to yield prodrugs are well-known in the art. For example, a hydroxyl functional group may be masked as a sulfonate, ester or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed in vivo to provide the amino group. A carboxyl group may be masked as an ester (including silyl esters and thioesters), amide or hydrazide promoiety, which may be hydrolyzed in vivo to provide the carboxyl group. Other specific examples of suitable progroups and their respective promoieties will be apparent to those of skill in the art.

“Progroup” refers to a type of protecting group that, when used to mask a functional group within an active 2,4-pyrimidinediamine drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃.

“Fc Receptor” refers to a member of the family of cell surface molecules that binds the Fc portion (containing the specific constant region) of an immunoglobulin. Each Fc receptor binds immunoglobulins of a specific type. For example the Fcα receptor (“FcαR”) binds IgA, the FcεR binds IgE and the FcγR binds IgG.

The FcαR family includes the polymeric Ig receptor involved in epithelial transport of IgA/IgM, the mycloid specific receptor RcαRI (also called CD89), the Fcα/μR and at least two alternative IgA receptors (for a recent review see Monteiro & van de Winkel, 2003, Annu. Rev. Immunol, advanced e-publication. The FcαRI is expressed on neutrophils, eosinophils, moncytes/macrophages, dendritic cells and kupfer cells. The FcαRI includes one alpha chain and the FcR gamma homodimer that bears an activation motif (ITAM) in the cytoplasmic domain and phosphorylates Syk kinase.

The FcεR family includes two types, designated FcεRI and FcεRII (also known as CD23). FcεRI is a high affinity receptor (binds IgE with an affinity of about 10¹⁰ M⁻¹) found on mast, basophil and eosinophil cells that anchors monomeric IgE to the cell surface. The FcεRI possesses one alpha chain, one beta chain and the gamma chain homodimer discussed above. The FcεRII is a low affinity receptor expressed on mononuclear phagocytes, B lymphocytes, eosinophils and platelets. The FcεRII comprises a single polypeptide chain and does not include the gamma chain homodimer.

The FcγR family includes three types, designated FcγRI (also known as CD64), FcγRII (also known as CD32) and FcγRIII (also known as CD16). FcγRI is a high affinity receptor (binds IgG1 with an affinity of 108M−1) found on mast, basophil, mononuclear, neutrophil, eosinophil, deudritic and phagocyte cells that anchors nomomeric IgG to the cell surface. The FcγRI includes one alpha chain and the gamma chain dimer shared by FcαRI and FcεRI.

The FcγRII is a low affinity receptor expressed on neutrophils, monocytes, eosinophils, platelets and B lymphocytes. The FcγRII includes one alpha chain, and does not include the gamma chain homodimer discussed above.

The FcγRIII is a low affinity (bindes IgG1 with an affinity of 5×10⁵ M⁻¹) expressed on NK, eosinophil, macrophage, neutrophil and mast cells. It comprises one alpha chain and the gamma homodimer shared by FcαRI, FcεRI and FcγRI.

Skilled artisans will recognize that the subunit structure and binding properties of these various Fc receptors, cell types expressing them, are not completely characterized. The above discussion merely reflects the current state-of-the-art regarding these receptors (see, e.g., Immunobiology: The Immune System in Health & Disease, 5th Edition, Janeway et al., Eds, 2001, ISBN 0-8153-3642-x, FIG. 9.30 at pp. 371), and is not intended to be limiting with respect to the myriad receptor signaling cascades that can be regulated with the compounds described herein.

“Fc Receptor-Mediated Degranulation” or “Fc Receptor-Induced Degranulation” refers to degranulation that proceeds via an Fc receptor signal transduction cascade initiated by crosslinking of an Fc receptor.

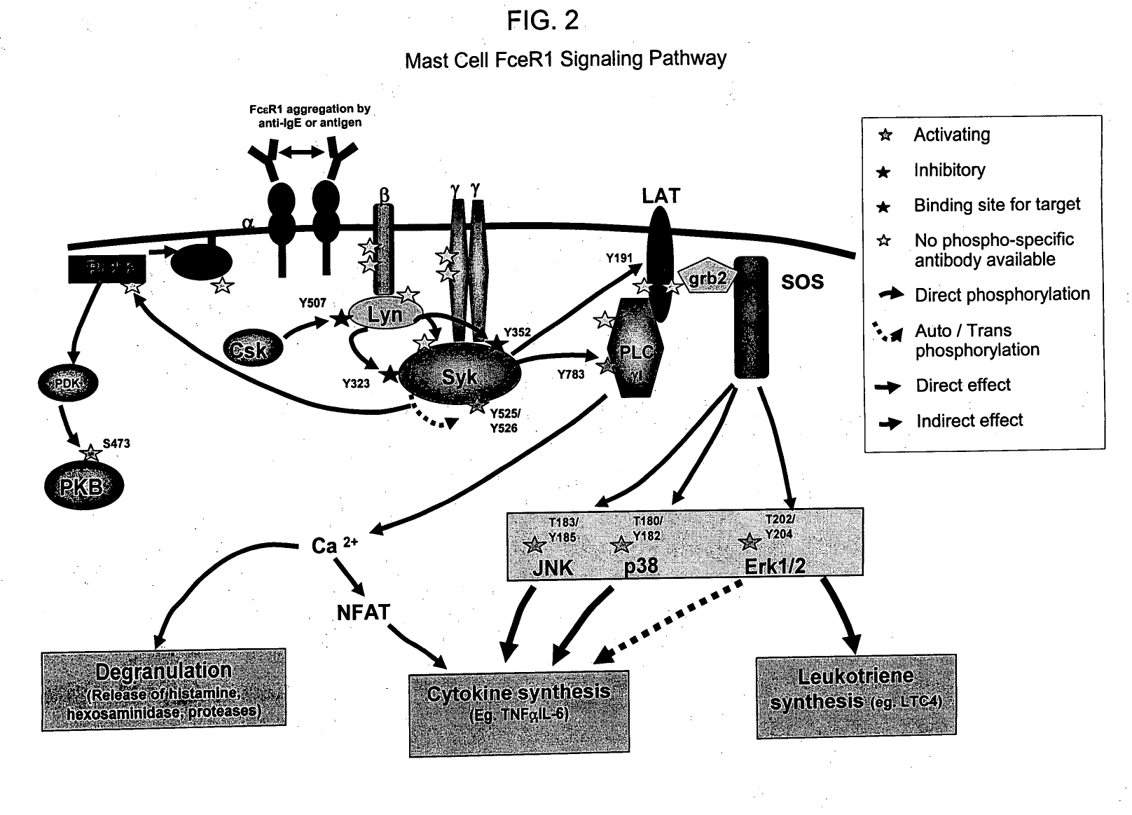

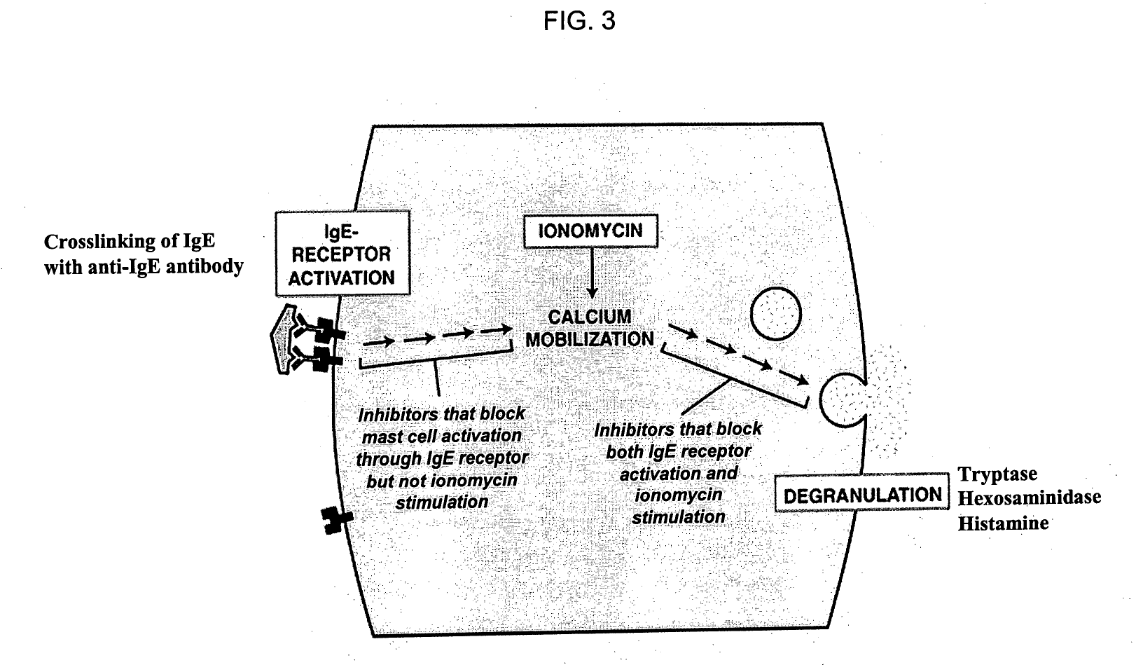

“IgE-Induced Degranulation” or “FcεRI-Mediated Degranulation” refers to degranulation that proceeds via the IgE receptor signal transduction cascade initiated by crosslinking of FcεR1-bound IgE. The crosslinking may be induced by an IgE-specific allergen or other multivalent binding agent, such as an anti-IgE antibody. Referring to FIG. 2, in mast and/or basophil cells, the FcεRI signaling cascade leading to degranulation may be broken into two stages: upstream and downstream. The upstream stage includes all of the processes that occur prior to calcium ion mobilization (illustrated as “Ca²⁺” in FIG. 2; see also FIG. 3). The downstream stage includes calcium ion mobilization and all processes downstream thereof. Compounds that inhibit FcεRI-mediated degranulation may act at any point along the FcεRI-mediated signal transduction cascade. Compounds that selectively inhibit upstream FcεRI-mediated degranulation act to inhibit that portion of the FcεRI signaling cascade upstream of the point at which calcium ion mobilization is induced. In cell-based assays, compounds that selectively inhibit upstream FcεRI-mediated degranulation inhibit degranulation of cells such as mast or basophil cells that are activated or stimulated with an IgE-specific allergen or binding agent (such as an anti-IgE antibody) but do not appreciably inhibit degranulation of cells that are activated or stimulated with degranulating agents that bypass the FcεRI signaling pathway, such as, for example the calcium ionophores ionomycin and A23187.

“IgG-Induced Degranulation” or “FcγRI-Mediated Degranulation” refers to degranulation that proceeds via the FcγRI signal transduction cascade initiated by crosslinking of FcγRI-bound IgG. The crosslinking may be induced by an IgG-specific allergen or another multivalent binding agent, such as an anti-IgG or fragment antibody. Like the FcεRI signaling cascade, in mast and basophil cells the FcγRI signaling cascade also leads to degranulation which may be broken into the same two stages: upstream and downstream. Similar to FcεRI-mediated degranulation, compounds that selectively inhibit upstream FcγRI-mediated degranulation act upstream of the point at which calcium ion mobilization is induced. In cell-based assays, compounds that selectively inhibit upstream FcγRI-mediated degranulation inhibit degranulation of cells such as mast or basophil cells that are activated or stimulated with an IgG-specific allergen or binding agent (such as an anti-IgG antibody or fragment) but do not appreciably inhibit degranulation of cells that are activated or stimulated with degranulating agents that bypass the FcγRI signaling pathway, such as, for example the calcium ionophores ionomycin and A23187.

“Ionophore-Induced Degranulation” or “Ionophore-Mediated Degranulation” refers to degranulation of a cell, such as a mast or basophil cell, that occurs upon exposure to a calcium ionophore such as, for example, ionomycin or A23187.

“Syk Kinsase” refers to the well-known 72 kDa non-receptor (cytoplasmic) spleen protein tyrosine kinase expressed in B-cells and other hematopoetic cells. Syk kinase includes two consensus Src-homology 2 (SH2) domains in tandem that bind to phosphorylated immunoreceptor tyrosine-based activation motifs (“ITAMs”), a “linker” domain and a catalytic domain (for a review of the structure and function of Syk kinase see Sada et al., 2001, J. Biochem. (Tokyo) 130:177-186); see also Turner et al., 2000, Immunology Today 21:148-154). Syk kinase has been extensively studied as an effector of B-cell receptor (BCR) signaling (Turner et al., 2000, supra). Syk kinase is also critical for tyrosine phosphorylation of multiple proteins that regulate important pathways leading from immunoreceptors, such as Ca2+ mobilization and mitogen-activated protein kinase (MAPK) cascades (see, e.g., FIG. 2) and degranulation. Syk kinase also plays a critical role in integrin signaling in neutrophils (see, e.g., Mocsai et al. 2002, Immunity 16:547-558).

As used herein, Syk kinase includes kinases from any species of animal, including but not limited to, homosapiens, simian, bovine, porcine, rodent, etc., recognized as belonging to the Syk family. Specifically included are isoforms, splice variants, allelic variants, mutants, both naturally occurring and man-made. The amino acid sequences of such Syk kinases are well known and available from GENBANK. Specific examples of mRNAs encoding different isoforms of human Syk kinase can be found at GENBANK accession no. gi|21361552|ref|NM_{—}003177.2|, gi|496899|emb|Z29630.1|HSSYKPTK[496899] and gi|15030258|gb|BC011399.1|BC011399[15030258], which are incorporated herein by reference.

Skilled artisans will appreciate that tyrosine kinases belonging to other families may have active sites or binding pockets that are similar in three-dimensional structure to that of Syk. As a consequence of this structural similarity, such kinases, referred to herein as “Syk mimics,” are expected to catalyze phosphorylation of substrates phosphorylated by Syk. Thus, it will be appreciated that such Syk mimics, signal transduction cascades in which such Syk mimics play a role and biological responses effected by such Syk mimics and Syk mimic-dependent signaling cascades may be regulated, and in particular inhibited, with the 2,4-pyrimidinediamine compounds described herein.

“Syk-Dependent Signaling Cascade” refers to a signal transduction cascade in which Syk kinase plays a role. Non-limiting examples of such Syk-dependent signaling cascades include the FcαRI, FcεRI, FcγRI, FcγRIII, BCR and integrin signaling cascades.

The present invention pertains to methods of synthesizing a 2,4-pyrimidinediamine compounds according to structural formula (I): 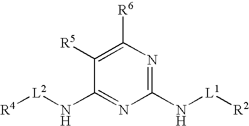
and salts, hydrates, solvates, N-oxides and prodrugs thereof, wherein:
L¹ and L² are each, independently of one another, selected from the group consisting of a direct bond and a linker;
R² is selected from the group consisting of (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;
R⁵ is selected from the group consisting of R⁶, (C1-C6) alkyl optionally substituted with one or more of the same or different R⁸ groups, (C1-C4) alkanyl optionally substituted with one or more of the same or different R⁸ groups, (C2-C4) alkenyl optionally substituted with one or more of the same or different R⁸ groups and (C2-C4) alkynyl optionally substituted with one or more of the same or different R⁸ groups;
each R⁶ is independently selected from the group consisting of hydrogen, an electronegative group, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CF₃, —CH₂CF₃, —CF₂CF₃, —CN, —NC, —OCN, —SCN, —NO, —NO₂, —N₃, —S(O)R^{d}, —S(O)₂R^{d}, —S(O)₂OR^{d}, —S(O)NR^{c}R^{c}; —S(O)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙNR^{c}R^{c} and —[NHC(NH)]ₙNR^{c}R^{c}, (C5-C10) aryl optionally substituted with one or more of the same or different R⁸ groups, phenyl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups and 6-16 membered heteroarylalkyl optionally substituted with one or more of the same or different R⁸ groups;
R⁸ is selected from the group consisting of R^{a}, R^{b}, R^{a} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₙC(O)NH—(CH₂)ₘR^{b}, —NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R^{a} is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b} is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}—C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c} is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each R^{d} is independently a protecting group or R^{a};
each m is independently an integer from 1 to 3; and
each n is independently an integer from 0 to 3.

In the compounds prepared by the methods of the invention, having structural formula (I), L¹ and L² represent, independently of one another, a direct bond or a linker. Thus, as will be appreciated by skilled artisans, the substituents R² and/or R⁴ may be bonded either directly to their respective nitrogen atoms or, alternatively, spaced away from their respective nitrogen atoms by way of a linker. The identity of the linker is not critical and typical suitable linkers include, but are not limited to, (C1-C6) alkyldiyls, (C1-C6) alkanos and (C1-C6) heteroalkyldiyls, each of which may be optionally substituted with one or more of the same or different R⁸ groups, where R⁸ is as previously defined for structural formula (I). In a specific embodiment, L¹ and L² are each, independently of one another, selected from the group consisting of a direct bond, (C1-C3) alkyldiyl optionally substituted with one or more of the same or different R^{a}, suitable R^{b} or R⁹ groups and 1-3 membered heteroalkyldiyl optionally substituted with one or more of the same or different R^{a}, suitable R^{b} or R⁹ groups, wherein R⁹ is selected from the group consisting of (C1-C3) alkyl, —OR^{a}, —C(O)OR^{a}, (C5-C10) aryl optionally substituted with one or more of the same or different halogens, phenyl optionally substituted with one or more of the same or different halogens, 5-10 membered heteroaryl optionally substituted with one or more of the same or different halogens and 6 membered heteroaryl optionally substituted with one or more of the same or different halogens; and R^{a} and R^{b} are as previously defined for structural formula (I). Specific R⁹ groups that may be used to substitute L¹ and L² include —OR^{a}, —C(O)OR^{a}, phenyl, halophenyl and 4-halophenyl, wherein R^{a} is as previously defined for structural formula (I).

In another specific embodiment, L¹ and L² are each, independently of one another, selected from the group consisting of methano, ethano and propano, each of which may be optionally monosubstituted with an R⁹ group, where R⁹ is as previously defined above.

In all of the above embodiments, specific R^{a} groups that may be included in R⁹ groups are selected from the group consisting of hydrogen, (C1-C6) alkyl, phenyl and benzyl.

In still another specific embodiment, L¹ and L² are each a direct bond such that the 2,4-pyrimidinediamine compounds prepared by the methods of the invention are compounds according to structural formula (Ia): 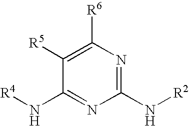
including salts, hydrates, solvates and N-oxides thereof, wherein R², R⁴, R⁵ and R⁶ are as previously defined for structural formula (I). Additional specific embodiments of the 2,4-pyrimidinediamine compounds prepared by the methods of the invention are described below.

In a first synthesis for the preparation of compounds of structural formulae (I) and (Ia), R², R⁴, R⁵, R⁶, L¹ and L² are as previously defined for their respective structures (I) and (Ia), with the proviso that R² is 3,4,5-trimethoxyphenyl, 3,4,5-tri (C1-C6) alkoxyphenyl or 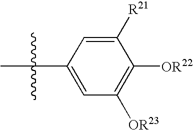
where R²¹, R²² and R²³ are as defined for R¹, R² and R³, respectively as in U.S. Pat. No. 6,235,746, the disclosure of which is incorporated by reference. In a specific embodiment of this first embodiment, R²¹ is hydrogen, halo, straight-chain or branched (C1-C6) alkyl optionally substituted with one or more of the same or different R²⁵ groups, hydroxyl, (C1-C6) alkoxy optionally substituted with one or more of the same or different phenyl or R²⁵ groups, thiol (—SH), (C1-C6) alkylthio optionally substituted with one or more of the same or different phenyl or R²⁵ groups, amino (—NH₂), —NHR²⁶ or —NR²⁶R²⁶; R²² and R²³ are each, independently of one another, a (C1-C6) straight-chain or branched alkyl optionally substituted with one or more of the same or different R²⁵ groups; R²⁵ is selected from the group consisting of halo, hydroxyl, (C1-C6) alkoxy, thiol, (C1-C6) alkylthio, (C1-C6) alkylamino and (C1-C6) dialkylamino; and each R²⁶ is independently a (C1-C6) alkyl optionally substituted with one or more of the same or different phenyl or R²⁵ groups or a —C(O)R²⁷, where R²⁷ is a (C1-C6) alkyl optionally substituted with one or more of the same or different phenyl or R²⁵ groups.

In another specific embodiment of this first synthesis, R²¹ is methoxy optionally substituted with one or more of the same or different halo groups and/or R²² and R²³ are each, independently of one another, a methyl or ethyl optionally substituted with one or more of the same or different halo groups.

In a second synthesis of the compounds of structural formulae (I) and (Ia), R², R⁴, R⁵ and L² are as previously described for their respective structures (I) and (Ia), L¹ is a direct bond and R⁶ is hydrogen, with the proviso that R² is 3,4,5-trimethoxyphenyl, 3,4,5-tri (C1-C6) alkoxyphenyl or 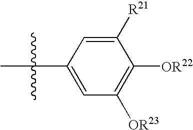
where R²¹, R²² and R²³ are as defined above, in connection with the first preparation.

In a third synthesis, 2,4-pyrimidinediamine compounds prepared by the method of the invention include structural formulae (I) and (Ia) include the following compounds:
N2,N4 bis(4 ethoxyphenyl) 5 fluoro 2,4 pyrimidinediamine;
N2,N4 bis(2 methoxyphenyl) 5 fluoro-2,4-pyrimidinediamine;
N2,N4 bis(4 methoxyphenyl) 5 fluoro 2,4 pyrimidinediamine;
N2,N4 bis(2 chlorophenyl) 5 fluoro 2,4 pyrimidinediamine;
N2,N4 bisphenyl 5 fluoro 2,4 pyrimidinediamine;
N2,N4 bis(3 methylphenyl) 5 fluoro 2,4 pyrimidinediamine;
N2,N4 bis(3 chlorophenyl) 5 fluoro 2,4 pyrimidinediamine;
N2,N4 bis(2,5 dimethylphenyl) 5 fluoro 2,4 pyrimidinediamine;
N2,N4 bis(3,4 dimethylphenyl) 5 fluoro 2,4 pyrimidinediamine;
N2,N4 bis(4 chlorophenyl) 5 fluoro 2,4 pyrimidinediamine;
N2,N4 bis(2,4 dimethylphenyl) 5 fluoro 2,4 pyrimidinediamine;
N2,N4 bis(3-bromophenyl)-5-fluoro-2,4-pyrimidinediamine;
N2,N4 bis(phenyl) 5 fluoro 2,4 pyrimidinediamine;
N2,N4 bis(morpholino) 5 fluoro 2,4 pyrimidinediamine; and
N2,N4-bis[(3-chloro-4-methoxyphenyl)]-5-fluoro-2,4-pyrimidinediamine.

In a fourth synthetic method of the invention, the compounds of structural formulae (I) and (Ia) include compounds according to the following structural formula (Ib): 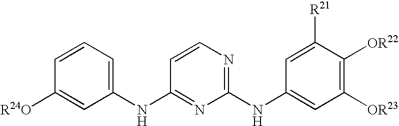
wherein R²⁴ is (C1-C6) alkyl; and R²¹, R²² and R²³ are as previously defined in connection with the first embodiment.

In a fifth synthetic method of the invention, the compounds of structural formulae (I) and (Ia) include the compounds described in Examples 1-141 of U.S. Pat. No. 6,235,746, the disclosure of which is incorporated herein by reference.

In a sixth synthetic method of the invention, the compounds of structural formulae (I) and (Ia) include compounds defined by formula (1) or formula 1(a) of U.S. Pat. No. 6,235,746 (see, e.g., the disclosure at Col. 1, line 48 through Col. 7, line 49 and Col. 8, lines 9-36, which is incorporated by reference).

In a seventh synthetic method of the invention, the compounds of structural formulae (I) and (Ia) include compounds in which R⁵ is cyano or —C(O)NHR, where R is hydrogen or (C1-C6) alkyl, when R² is a substituted phenyl; R⁴ is a substituted or unsubstituted (C₁-C₆) alkyl, (C3-C8) cycloalkyl, 3-8 membered cycloheteralkyl or 5-15 membered heteroaryl; and R⁶ is hydrogen.

In an eighth synthetic method of the invention, the compounds of structural formulae (I) and (Ia) include the compounds defined by formulae (I) and (X) of WO 02/04429 or any compound disclosed in WO 02/04429, the disclosure of which is incorporated herein by reference.

In a ninth synthetic method of the invention, the compounds include structural formulae (I) and (Ia), when R⁵ is cyano or —C(O)NHR, where R is hydrogen or (C1-C6) alkyl; and R⁶ is hydrogen, then R² is other than a substituted phenyl group.

In a tenth synthetic method of the invention, the compounds of structural formulae (I) and (Ia) include compounds in which R² and R⁴ are each independently a substituted or unsubstituted pyrrole or indole ring which is attached to the remainder of the molecule via its ring nitrogen atom.

In an eleventh synthetic method of the invention, the compounds of structural formulae (I) and (Ia) include compounds defined by formulae (I) and (IV) of U.S. Pat. No. 4,983,608 or any compound disclosed in U.S. Pat. No. 4,983,608, the disclosure of which is incorporated herein by reference.

Those of skill in the art will appreciate that in the compounds of formulae (I) and (Ia), R² and R⁴ may be the same or different, and may vary broadly. When R² and/or R⁴ are optionally substituted rings, such as optionally substituted cycloalkyls, cycloheteroalkyls, aryls and heteroaryls, the ring may be attached to the remainder of the molecule through any available carbon or heteroatom. The optional substituents may be attached to any available carbon atoms and/or heteroatoms.

In a twelfth synthetic method of the invention, provide compounds having structural formulae (I) and (Ia), when R² and/or R⁴ is an optionally substituted phenyl or an optionally substituted (C5-C15) aryl, subject to the provisos that (1) when R⁶ is hydrogen, then R² is 3,4,5-trimethoxyphenyl or 3,4,5-tri (C1-C6) alkoxyphenyl; (2) when R² is a 3,4,5-trisubstituted phenyl, then the substituents at the 3- and 4-positions are simultaneously methoxy or (C1-C6) alkoxy; or (3) when R⁶ is hydrogen and R⁴ is (C1-C6) alkyl, (C3-C8) cycloalkyl, 3-8 membered cycloheteroalkyl or 5-15 membered heteroaryl, then R⁵ is cyano. Alternatively, R² is subject to the provisos described in connection with the first or second embodiments. The optionally substituted aryl or phenyl group may be attached to the remainder of the molecule through any available carbon atom. Specific examples of optionally substituted phenyls include phenyls that are optionally mono-, di- or tri-substituted with the same or different R⁸ groups, where R⁸ is as previously defined for structural formula (I) and subject to the above provisos. When the phenyl is mono-substituted, the R⁸ substituent may be positioned at either the ortho, meta or para position. When positioned at the ortho, meta or para position, R⁸ is preferably selected from the group consisting of (C1-C10) alkyl, (C1-C10) branched alkyl, —OR^{a} optionally substituted with one or more of the same or different R^{b} groups, —O—C(O)OR^{a}, —O—(CH₂)ₘ—C(O)OR^{a}, —C(O)OR^{a}, —O—(CH₂)ₘ—NR^{c}R^{c}, —O—C(O)NR^{c}R^{c}, —O—(CH₂)ₘ—C(O)NR^{c}R^{c}, —O—C(NH)NR^{c}R^{c}, —O—(CH₂)ₘ—C(NH)NR^{c}R^{c} and —NH—(CH₂)ₘ—NR^{c}R^{c}, where m, R^{a} and R^{c} are as previously defined for structural formula (I). In one embodiment of these compounds, —NR^{c}R^{c} is a 5-6 membered heteroaryl, which optionally includes one or more of the same or different additional heteroatoms. Specific examples of such 5-6 membered heteroaryls include, but are not limited to, oxadiazolyl, triazolyl, thiazolyl, oxazolyl, tetrazolyl and isoxazolyl.

In another synthetic method preparation of these compounds, —NR^{c}R^{c} is a 5-6 membered saturated cycloheteroalkyl ring, which optionally includes one or more of the same or different heteroatoms. Specific examples of such cycloheteroalkyls include, but are not limited to, pyrrolidinyl, pyrazolidinyl, imidazolidinyl, piperidinyl, piperazinyl and morpholinyl.

In still another synthetic preparation of these compounds, each R^{a} is independently a (C1-C6) alkyl and/or each —NR^{c}R^{c} is —NHR^{a}, where R^{a} is a (C1-C6) alkyl. In one specific embodiment, R⁸ is —O—CH₂—C(O)NHCH₃. In another specific embodiment R⁸ is H.

When the phenyl is di-substituted or tri-substituted, the R⁸ substituents may be positioned at any combination of positions. For example, the R⁸ substituents may be positioned at the 2,3-, 2,4-, 2,5-, 2,6-, 3,4-, 3,5-, 2,3,4-, 2,3,5-, 2,3,6-, 2,4,5-, 2,4,6-, 2,5,6- or 3,4,5-positions. In one embodiment of compounds including a disubstituted phenyl, the substituents are positioned other than 3,4. In another embodiment they are positioned 3,4. In one embodiment of compounds including a trisubstituted phenyl, the substituents are positioned other than 3,4,5 or, alternatively, no two of the substituents are positioned 3,4. In another embodiment, the substituents are positioned 3,4,5.

Specific examples of R⁸ substituents in such di- and trisubstituted phenyls include the various R⁸ substituents described above in connection with the ortho, meta and para substituted phenyls.

In another specific embodiment, compounds that can be prepared by the present method include R⁸ substituents useful for substituting such di- and trisubstituted phenyls and include (C1-C6) alkyl, (C1-C6) alkoxy, methoxy, halo, chloro, (C1-C6) perhaloalkyl, —CF₃, (C1-C6) perhaloalkoxy and —OCF₃. In a preferred embodiment, such R⁸ substituents are positioned 3,4 or 3,5. Specific examples of preferred di-substituted phenyl rings include 3-chloro-4-methoxy-phenyl, 3-methoxy-4-chlorophenyl, 3-chloro-4-trifluoromethoxy-phenyl, 3-trifluoromethoxy-4-chloro-phenyl, 3,4-dichloro-phenyl, 3,4-dimethoxyphenyl and 3,5-dimethoxyphenyl. Suitable examples include (1) when R⁴ is one of the above-identified phenyls, and R⁵ and R⁶ are each hydrogen, then R² can be 3,4,5-tri(C1-C6)alkoxyphenyl or 3,4,5-trimethoxyphenyl; (2) when R is 3,4-dimethoxyphenyl and R⁵ and R⁶ are each hydrogen, then R⁴ can be 3-(C1-C6)alkoxyphenyl, 3-methoxyphenyl, 3,4-di-(C1-C6) alkoxyphenyl or 3,4-dimethoxyphenyl; (3) when R⁴ is 3-chloro-4-methoxyphenyl and R⁵ is halo or fluoro, and optionally R⁶ is hydrogen, then R² can be 3-chloro-4-(C1-C6)alkoxyphenyl or 3-chloro-4-methoxyphenyl; (4) when R⁴ is 3,4-dichlorophenyl, R⁵ is hydrogen, (C1-C6) alkyl, methyl, halo or chloro and optionally R⁶ is hydrogen, then R² can be a phenyl mono substituted at the para position with a (C1-C6) alkoxy group which is optionally substituted with one or more of the same or different R^{b}, —OH or —NR^{c}R^{c} groups, where R^{b} and R^{c} are as previously described for structural formula (I); and/or (5) R² and/or R⁴ can be 3,4,5-tri(C1-C6)alkoxyphenyl or 3,4,5-trimethoxyphenyl, especially when R⁵ and R⁶ are each hydrogen.

In another synthetic method useful to prepare compounds including a trisubstituted phenyl, the trisubstituted phenyl has the formula: 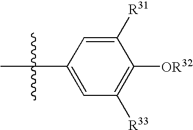
wherein: R³¹ is methyl or (C1-C6) alkyl; R³² is hydrogen, methyl or (C1-C6) alkyl; and R³³ is a halo group.

In a thirteenth synthetic method of the invention, when compounds include the structural formulae (I) and (Ia), R² and/or R⁴ is an optionally substituted heteroaryl. Typical heteroaryl groups according to this thirteenth embodiment comprise from 5 to 15, and more typically from 5 to 11 ring atoms, and include one, two, three or four of the same or different heteratoms or heteroatomic groups selected from the group consisting of N, NH, O, S, S(O) and S(O)₂. The optionally substituted heteroaryl may be attached to its respective C2 or C4 nitrogen atom or linker L¹ or L² through any available carbon atom or heteroatom, but is typically attached via a carbon atom. The optional substituents may be the same or different, and may be attached to any available carbon atom or heteroatom. In one embodiment of these compounds, R⁵ is other than bromo, nitro, trifluoromethyl, cyano or —C(O)NHR, where R is hydrogen or (C1-C6) alkyl. In another embodiment of these compounds, when R² and R⁴ are each a substituted or unsubstituted pyrrole or indole, then the ring is attached to the remainder of the molecule via a ring carbon atom. In still another embodiment of compounds including an optionally substituted heteroaryl group, the heteroaryl is unsubstituted or substituted with from one to four of the same or different R⁸ groups, where R⁸ is as previously defined for structural formula (I). Specific examples of such optionally substituted heteroaryls include, but are not limited to, the following heteroaryl groups: 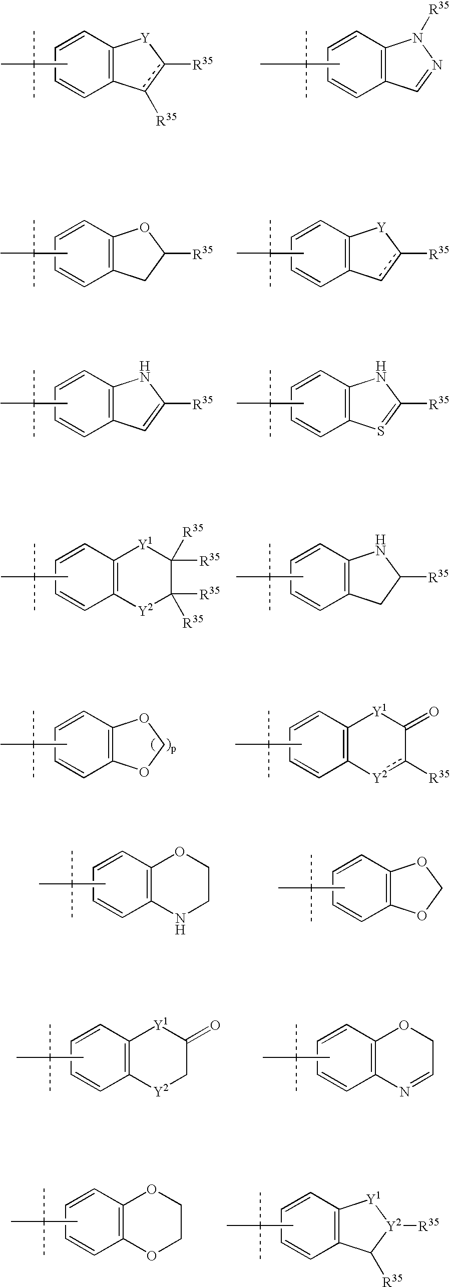 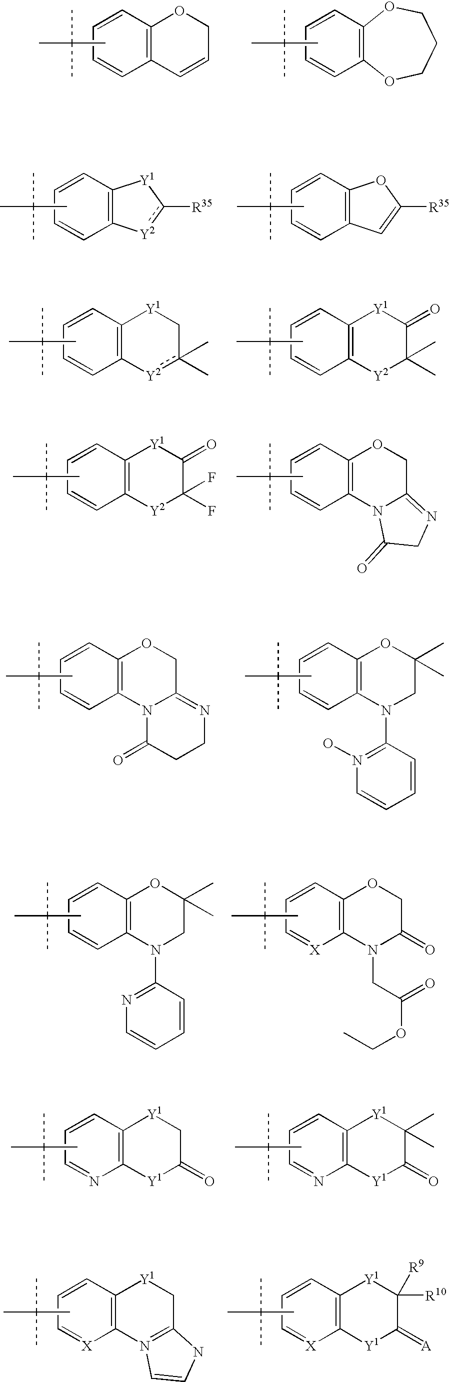 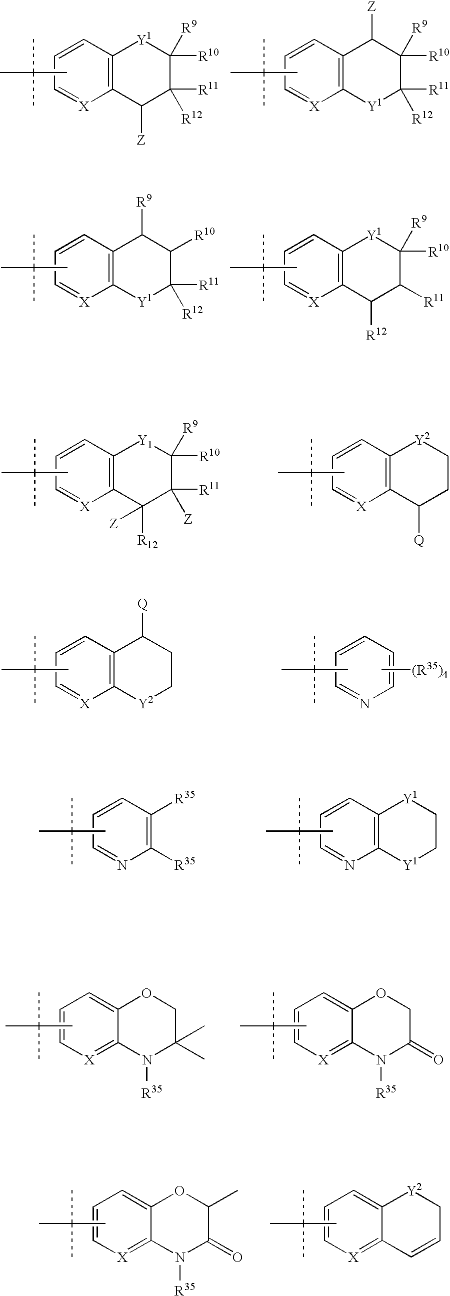 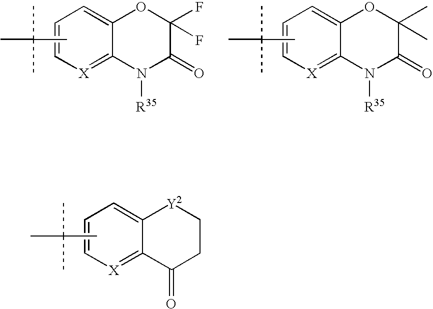
wherein:
p is an integer from one to three;
each [symbol] independently represents a single bond or a double bond;
R³⁵ is hydrogen or R⁸, where R⁸ is as previously defined for structural formula (I);
X is selected from the group consisting of CH, N and N—O;
each Y is independently selected from the group consisting of O, S and NH;
each Y¹ is independently selected from the group consisting of O, S, SO, SO₂, SONR³⁶, NH and NR³⁷;
each Y² is independently selected from the group consisting of CH, CH₂, O, S, N, NH and NR³⁷;
R³⁶ is hydrogen or alkyl;
R³⁷ is selected from the group consisting of hydrogen and a progroup, preferably hydrogen or a progroup selected from the group consisting of aryl, arylalkyl, heteroaryl, R^{a}, R^{b}—CR^{a}R^{b}—O—C(O)R⁸, —CR^{a}R^{b}—O—PO(OR⁸)₂, —CH₂—O—PO(OR⁸)₂, —CH₂—PO(OR⁸)₂, —C(O)—CR^{a}R^{b}—N(CH₃)₂, —CR^{a}R^{b}—O—C(O)—CR^{a}R^{b}—N(CH₃)₂, —C(O)R⁸, —C(O)CF₃ and —C(O)—NR⁸—C(O)R⁸;
   A is selected from the group consisting of O, NH and NR³⁸;
   R³⁸ is selected from the group consisting of alkyl and aryl;
   R⁹, R¹⁰, R¹¹ and R¹² are each, independently of one another, selected from the group consisting of alkyl, alkoxy, halogen, haloalkoxy, aminoalkyl and hydroxyalkyl, or, alternatively, R⁹ and R¹⁰ and/or R¹¹ and R¹² are taken together form a ketal;
   each Z is selected from the group consisting of hydroxyl, alkoxy, aryloxy, ester, carbamate and sulfonyl;
   Q is selected from the group consisting of —OH, OR⁸, —NR^{c}R^{c}, —NHR³⁹—C(O)R⁸, —NHR³⁹—C(O)OR⁸, —NR³⁹—CHR⁴⁰—R^{b}, —NR³⁹—(CH₂)ₘ—R^{b} and —NR³⁹—C(O)—CHR⁴⁰—NR^{c}R^{c};
R³⁹ and R⁴⁰ are each, independently of one another, selected from the group consisting of hydrogen, alkyl, aryl, alkylaryl; arylalkyl and NHR⁸; and
   R^{a}, R^{b} and R^{c} are as previously defined for structural formula (I). Preferred Rb substitutents for Q are selected from —C(O)OR⁸, —O—C(O)R⁸, —O—P(O)(OR⁸)₂ and —P(O)(OR⁸)₂.

In one embodiment of the above-depicted heteroaryls, as well as other 5-15 membered heteroaryls according to this embodiment of the invention, each R⁸ is independently selected from the group consisting of R^{d}, —NR^{c}R^{c}, —(CH₂)ₘ—NR^{c}R^{c}, —C(O)NR^{c}R^{c}, —(CH₂)ₘ—C(O)NR^{c}R^{c}, —C(O)OR^{d}, —(CH₂)ₘ—C(O)OR^{d} and —(CH₂)ₘ—OR^{d}, where m, R^{c} and R^{d} are as previously defined for structural formula (I).

In a specific synthesis, R^{d} and/or R^{c} is selected from the group consisting of R^{a} and (C3-C8) cycloalkyl optionally substituted with one or more of the same or different hydroxyl, amino or carboxyl groups.

In another synthesis of the above-depicted heteroaryls, each R³⁵ is hydrogen or (C1-C6) ethyl or methyl.

In still another synthesis of the above-depicted heteroaryls, the aromatic ring connectivity is either at the 5 or 6 position. It should be understood that either R² or R⁴ can utilize the heteroaryl groups discussed throughout this specification.

In a fourteenth synthesis of the compounds of structural formulae (I) and (Ia), R² and R⁴ are each, independently of one another, an optionally substituted phenyl, aryl or heteroaryl, with the provisos that: (1) when L¹ is a direct bond and R⁶ and optionally R⁵ is hydrogen, then R² can be 3,4,5-trimethoxyphenyl or 3,4,5-tri(C1-C6) alkoxyphenyl; (2) when L¹ and L² are each a direct bond, R⁶ is hydrogen and R⁵ is halo, then R² and R⁴ can each simultaneously be 3,4,5-trimethoxyphenyl or 3,4,5-tri(C1-C6) alkoxyphenyl; (3) when R⁴ is 3-methoxyphenyl or 3-(C1-C6) alkoxyphenyl and R² is a 3,4,5-trisubstituted phenyl, the substituents positioned at the 3 and 4 positions can be both simultaneously methoxy or (C1-C6) alkoxy; (4) when R² is a substituted phenyl and R⁶ is hydrogen, then R⁵ can be cyano or —C(O)NHR, where R is hydrogen or (C1-C6) alkyl; and/or (5) when R² and R⁴ are each independently a substituted or unsubstituted pyrrole or indole, then the pyrrole or indole is attached to the remainder of the molecule via a ring carbon atom. Alternatively, R² is subject to the provisos described in connection with the first or second embodiment.

In a fourteenth synthesis of the invention, the R² and R⁴ substituents may be the same or different. Specific optionally substituted phenyl, aryl and/or heteroaryls include those illustrated above in connection with the twelfth and thirteenth embodiments.

In a fifteenth synthesis of the compounds of structural formulae (I) and (Ia), including the above-described first through fourteenth embodiments thereof, R⁶ is hydrogen and R⁵ is an electronegative group. As will be recognized by skilled artisans, electronegative groups are atoms or groups of atoms that have a relatively great tendency to attract electrons to themselves. Specific examples of electronegative groups according to this fourteenth embodiment include, but are not limited to, —CN, —NC, —NO₂, halo, bromo, chloro, fluoro, (C1-C3) haloalkyl, (C1-C3) perhaloalkyl, (C1-C3) fluoroalkyl, (C1-C3) perfluoroalkyl, —CF₃, (C1-C3) haloalkoxy, (C1-C3) perhaloalkoxy, (C1-C3) fluoroalkoxy, (C1-C3) perfluoroalkoxy, —OCF₃, —C(O)R^{a}, —C(O)OR^{a}, —C(O)CF₃ and —C(O)OCF₃. In a specific embodiment, the electronegative group is a halogen-containing electronegative group, such as —OCF₃, —CF₃, bromo, chloro or fluoro. In another specific embodiment, R⁵ is fluoro, subject to the proviso that the compound is not any compound according to the third embodiment.

In a sixteenth synthesis, the compounds of structural formulae (I) and (Ia) are compounds according to structural formula (Ib): 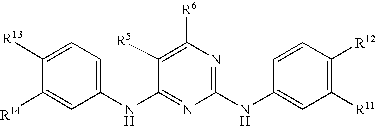
and salts, hydrates, solvates and N-oxides thereof, wherein R¹¹, R¹², R¹³ and R¹⁴ are each, independently of one another, selected from the group consisting of hydrogen, hydroxy, (C1-C6) alkoxy and —NR^{c}R^{c}; and R⁵, R⁶ and R^{c} are as previously defined for structural formula (I), with the proviso that when R¹³, R⁵ and R⁶ are each hydrogen, then R¹¹ and R¹² are not simultaneously methoxy, (C1-C6) alkoxy or (C1-C6) haloalkoxy.

In a seventeenth synthesis, the compounds of structural formulae (I) and (Ia) are compounds according to structural formula (Ic): 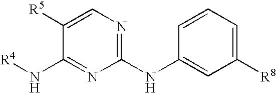
and salts, hydrates, solvates and N-oxides thereof, wherein:
R⁴ is selected from the group consisting of 5-10 membered heteroaryl and 3-hydroxyphenyl;
R⁵ is F or —CF₃; and
R⁸ is —O(CH₂)ₘ—R^{b}, where m and R^{b} are as previously defined for structural formula (I). In a specific embodiment, R⁸ is —O—CH₂—C(O)NH—CH₃ and/or R⁴ is a heteroaryl according to the thirteenth embodiment.

Those of skill in the art will appreciate that the synthetic preparations of the 2,4-pyrimidinediamine compounds described herein may include functional groups that can be masked with progroups to create prodrugs. Such prodrugs are usually, but need not be, pharmacologically inactive until converted into their active drug form. Indeed, many of the active 2,4-pyrimidinediamine compounds described in TABLE 1, of U.S. Ser. No. 10/335,543, the contents of which are incorporate herein by reference, include promoieties that are hydrolyzable or otherwise cleavable under conditions of use. For example, ester groups commonly undergo acid-catalyzed hydrolysis to yield the parent carboxylic acid when exposed to the acidic conditions of the stomach, or base-catalyzed hydrolysis when exposed to the basic conditions of the intestine or blood. Thus, when administered to a subject orally, 2,4-pyrimidinediamines that include ester moieties may be considered prodrugs of their corresponding carboxylic acid, regardless of whether the ester form is pharmacologically active. Referring to TABLE 1 of U.S. Ser. No. 10/335,543, numerous ester-containing 2,4-pyrimidinediamines of the invention are active in their ester, “prodrug” form.

For prodrugs prepared by the method of the invention, any available functional moiety may be masked with a progroup to yield a prodrug. Functional groups within the 2,4-pyrimidinediamine compounds that may be masked with progroups for inclusion in a promoiety include, but are not limited to, amines (primary and secondary), hydroxyls, sulfanyls (thiols), carboxyls, etc. Myriad progroups suitable for masking such functional groups to yield promoieties that are cleavable under the desired conditions of use are known in the art. All of these progroups, alone or in combinations, may be included in the prodrugs of the invention.

In one illustrative embodiment, the prodrugs prepared by the methods of the invention are compounds according to structural formula (I) in which R^{c} and R^{d} may be, in addition to their previously-defined alternatives, a progroup.

The hydrogens attached to N2 and N4 in the 2,4-pyrimidinediamines of structural formula (I) can be substituted with promoieties. As will be appreciated by skilled artisans, these nitrogens may be included in promoieties that, under conditions of use, cleave to yield 2,4-pyrimidinediamines according to structural formula (I). Thus, in another embodiment, the prodrugs of the invention are compounds according to structural formula (ii): 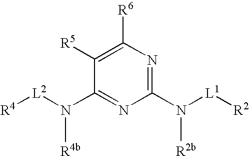
including salts, hydrates, solvates and N-oxides thereof, wherein:
R², R⁴, R⁵, R⁶, L¹ and L² are as previously defined for structural formula (I); and
R^{2b} and R^{4b} are each, independently of one another, a progroup. Specific examples of progroups according to this embodiment of the invention include, but are not limited to, (C1-C6) alkyl, —C(O)CH₃, —C(O)NHR³⁶ and —S(O)₂R³⁶, where R³⁶ is (C1-C6) alkyl, (C5-C15) aryl and (C3-C8) cycloalkyl.

In the prodrugs of structural formula (ii), the various substituents may be as described for the various first through twentieth embodiments previously described for the compounds of structural formulae (I) and (Ia), or combinations of such embodiments.

Those of skill in the art will appreciate that many of the compounds and prodrugs of the prepared by the method of the invention, as well as the various compound species specifically described and/or illustrated herein, may exhibit the phenomena of tautomerism, conformational isomerism, geometric isomerism and/or optical isomerism. For example, the compounds and prodrugs of the invention may include one or more chiral centers and/or double bonds and as a consequence may exist as stereoisomers, such as double-bond isomers (i.e., geometric isomers), enantiomers and diasteromers and mixtures thereof, such as racemic mixtures. As another example, the compounds and prodrugs of the invention may exist in several tautomeric forms, including the enol form, the keto form and mixtures thereof. As the various compound names, formulae and compound drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the compounds or prodrugs having one or more of the utilities described herein, as well as mixtures of these various different isomeric forms. In cases of limited rotation around the 2,4-pryimidinediamine core structure, atrop isomers are also possible and are also specifically included in the compounds prepared by the methods of the invention.

Moreover, skilled artisans will appreciate that when lists of alternative substituents include members which, owing to valency requirements or other reasons, cannot be used to substitute a particular group, the list is intended to be read in context to include those members of the list that are suitable for substituting the particular group. For example, skilled artisans will appreciate that while all of the listed alternatives for R^{b} can be used to substitute an alkyl group, certain of the alternatives, such as ═O, cannot be used to substitute a phenyl group. It is to be understood that only possible combinations of substituent-group pairs are intended.

The compounds and/or prodrugs prepared by the methods of the invention may be identified by either their chemical structure or their chemical name. When the chemical structure and the chemical name conflict, the chemical structure is determinative of the identity of the specific compound.

Depending upon the nature of the various substituents, the 2,4-pyrimidinediamine compounds and prodrugs prepared by the methods of the invention may be in the form of salts. Such salts include salts suitable for pharmaceutical uses (“pharmaceutically-acceptable salts”), salts suitable for veterinary uses, etc. Such salts may be derived from acids or bases, as is well-known in the art.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion) or coordinates with an organic base (e.g., ethanolamine, diethanolamine, triethanolamine, N-methylglucamine, morpholine, piperidine, dimethylamine, diethylamine, etc.).

The 2,4-pyrimidinediamine compounds prepared by methods of the invention, as well as the salts thereof, may also be in the form of hydrates, solvates and N-oxides, as are well-known in the art.

The compounds and prodrugs prepared by the methods of the invention may be synthesized using commercially available starting materials and/or starting materials prepared by conventional synthetic methods. Specific examples describing the synthesis of compounds of the invention, as well as intermediates therefore, are provided in the Examples section. All of the compounds of structural formulae (I), (Ia) and (ii) may be prepared by routine adaptation of these methods.

The compounds of the invention can be use for treatment of autoimmune diseases.

The present invention provide syntheses that include treating a compound according to structural formula (II) with a halogenating agent 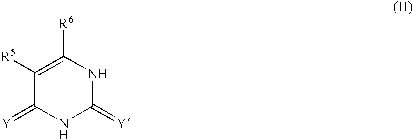
wherein Y and Y′ are each, independently of one another are selected from the group consisting of O and S and R⁵ and R⁶ are as defined above. For example, compound (II) is treated in a first step (a) with a halogenating agent, such as PX₅ (PCl₅, PBr₅), phosphorous oxyhalide (POCl₃, POBr₃) or mixtures thereof, at an elevated temperature, up to an including reflux conditions.

In one embodiment, compound (II) is treated with an excess of POCl₃ at a temperature between about 60° C. and about 150° C., more particularly between about 80° C. and about 120° C. and in particular, at about 110° C., for a period of time of between about 30 minutes to about 12 hours, more particularly between about 2 hours and about 10 hours, and in particular, for a period of about 8 hours and then cooled to room temperature. Excess PCl₅ is then added and heated at a temperature of between about 60° C. and about 150° C., more particularly between about 80° C. and about 120° C. and in particular, at reflux for a period of time, generally for a period of time of between about 30 minutes and about 18 hours, more particularly, between about 2 hours and about 14 hours, and in particular, for about 12 hours. The mixture can then been cooled to room temperature and poured into ice water with sodium chloride, causing the product (II) to precipitate from solution. The product can be collected by filtration for further processing.

In an alternative embodiment, compound (II) is treated with a halogenating agent or combination of halogenating agents in the presence of a tertiary amine solvent, such as an N,N-dialkylaniline. Suitable N,N-dialkylanilines include dimethylaniline, diethylaniline and the like. In one method, excess POX₃, i.e., POCl₃ can be removed by distillation with the product quenched in water and extracted into an organic solvent, such as methylene chloride, carbon tetrachloride, ethyl acetate or ether. Alternatively, the mixture of the POX₃, i.e., POCl₃ and product can be directly quenched in an acidic aqueous solution, such as 3 M HCl, and methylene chloride. The product of step (a) is compound (III) 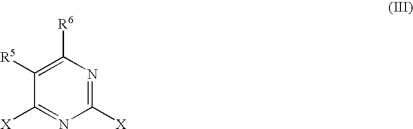
wherein each X is a halogen.

Compound (III) is extracted into the organic layer, i.e., methylene chloride layer, and can be isolated as an off-white solid in yields approaching 95%. Alternatively, compound (III) can be treated in step (b) without isolation or purification. Advantageously, the extraction solvent, such as methylene chloride, can be recycled and reused in additional preparative methods.

Use of the tertiary amine helps to scavenge HCl by-product from the reaction and eliminates the need for use of PX₅. PX₅ and/or POX₃ halogenating agents are difficult to quench. Use of the solvent also improved quenching of the excess halogenating agent(s). Typically, quenching of the reaction mixture without solvent present afforded an exothermic reaction that was difficult to control. Use of the solvent advantageously helped to reduce the exothermic nature of the quenching step.

In step (b), compound (III) is treated in a solvent at an elevated temperature with an equivalent of a compound according to structural formula (IV)
R⁴-L²-NH₂ (IV)
thereby forming a compound according to structural formula (V)

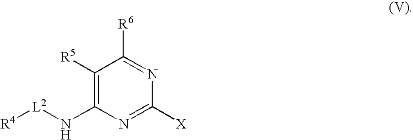

Typically the solvent used in step (b) is a C1-C7 straight chain or branched alcohol, such as methanol, ethanol, isopropyl alcohol, t-butyl alcohol, hexanol and the like. Generally the mixture in step (b) is heated over a temperature range of between about 60° C. and about 150° C., more particularly between about 80° C. and about 120° C. and in particular, at reflux conditions for a period of between about 30 minutes to about 12 hours, more particularly between about 2 hours and about 10 hours, and in particular, for a period of about 6 to about 8 hours. Compound V can be isolated and purified or can be treated in following step (c) without the need for isolation and purification.

In step (c), compound (V) is treated with an equivalent of a compound according to the structural formula (VI)
R²-L¹-NH₂ (VI)
in a solvent at an elevated temperature to form compound (I), wherein R², R⁴, R⁵, R⁶, L¹ and L² are as defined above.

Typically the solvent used in step (c) is also an alcohol, such as those described above, and for example include methanol, ethanol, isopropyl alcohol and the like. Generally the mixture in step (c) is heated over a temperature range of between about 60° C. and about 150° C., more particularly between about 80° C. and about 120° C. and in particular, at reflux conditions for a period of between about 30 minutes to about 12 hours, more particularly between about 2 hours and about 10 hours, and in particular, for a period of about 6 to about 8 hours.

In certain embodiments, steps (b) and (c) can be combined into one step, where compound (VI) is added to the reaction mixture after compound (IV) has been reacted with compound (III). It should be understood, that compound (VI) can be added in step (b) and compound (IV) can be added in step (c); they are interchangeable in terms of when the addition occurs and can be utilized to prepare compounds having varying R² and R⁴ substituents. Additionally, compounds (IV) and (VI) can be identical, providing a symmetrical 2,4-pyrimidinediamine compound.

In an alternative synthetic procedure, the reaction product of step (a), compound (III), is not isolated but can be exchanged with an alcohol, such as 2-propanol. The alcoholic solution can then be treated as described above in steps (b) and (c). The resultant HCL salt, can be easily collected, for example by filtration, rinsed with solvent, and isolated. This provides an advantage that the HCl salt can be rinsed to remove any impurities. The HCL salt of (I) can be converted into the free based by dissolving the salt in water and adjusting the pH to about 5.5 with a suitable base, such as sodium hydroxide. Isolation of the salt, followed by conversion, avoids purification by chromatography and/or recrystallization, which can be expensive and time consuming.

It should be understood that the temperature ranges, heating periods, solvents, etc. depend on the volume/size of the reaction and the time period desired. One skilled in the art would recognize that the various parameters can be modified to achieve a specific result, such as a decrease in a heating period by an increase in reaction temperature. These modification choices are within the skill of the ordinary artisan.

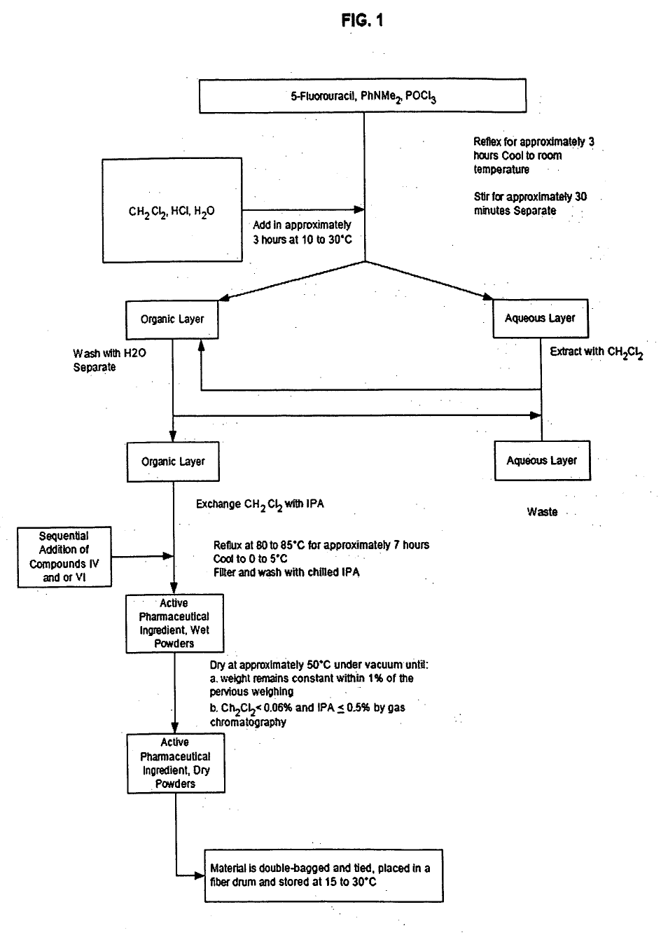

A representative synthetic method is provided in Scheme 1 and FIG. 1, wherein 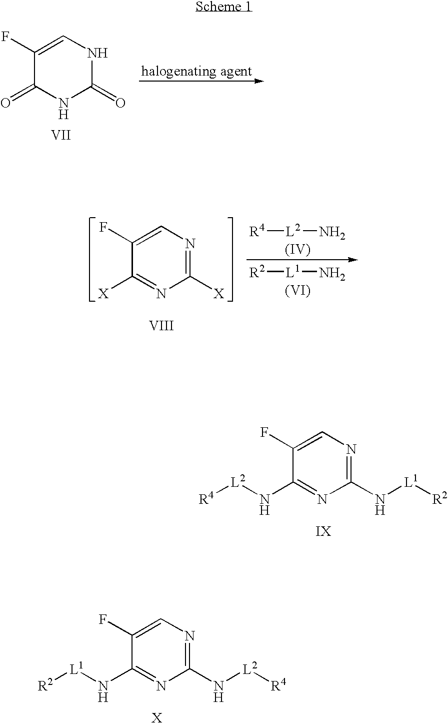
R², R⁴, L¹ and L² are as defined above.

In certain embodiments, L¹ and L² are direct bonds. It should be understood that 2 equivalents of (IV) or 2 equivalents of (VI) can be used in the synthetic method. Alternatively, (IV) or (VI) can be reacted in a first step and then the (VI) or (IV) can be reacted in a subsequent step to provide either (IX) or (X). One advantage of the synthesis of Scheme 1 is that intermediate (VIII) does not require isolation.

FIG. 1 is a flow chart for the preparation of compounds having formulae (IX) or (X), preferably with L¹ and L² being direct bonds. FIG. 1 demonstrates that the product dissolved in organic solvent, methylene chloride, can be exchanged with an alcohol, isopropanol. Advantageously, the intermediate can be treated with a suitable amine or amines to produce the final active pharmaceutical ingredient as the product.

### EXAMPLE

### Preparation of N2,N4-Bis(3-hydroxyphenyl)-5-fluoro-2,4-pyrimidinediamine, HCl salt

As shown in Scheme 2, a mixture of 5-fluorouracil (2000 g) (XI), N,N-dimethylaniline (3720 g) and phosphorus oxychloride (12 L) was refluxed under nitrogen for 3 hours. The resulting mixture was cooled to room temperature. Intermediate (XII) was not isolated but was used as follows. Dichloromethane, hydrochloric acid and water were added over approximately 3 hours between 10 to 30° C. The mixture was stirred for approximately 1 hour at 10 to 30° C. and then allowed to settle for approximately 30 minutes and separated. The aqueous layer was extracted with dichloromethane and combined with the organic layer. The combined organic layer was washed with water and the dicloromethane exchanged for isopropanol. 3-Aminophenol (4853 g) (XIII) was added and the mixture refluxed at 80 to 85° C. for approximately 7 hours. After cooling to 0 to 5° C., solid (XIV) was collected by filtration and washed with chilled isopropanol. The wet powder was dried under vacuum until a constant weight was obtained. The final product (XIV) was obtained as a solid (1657 g).

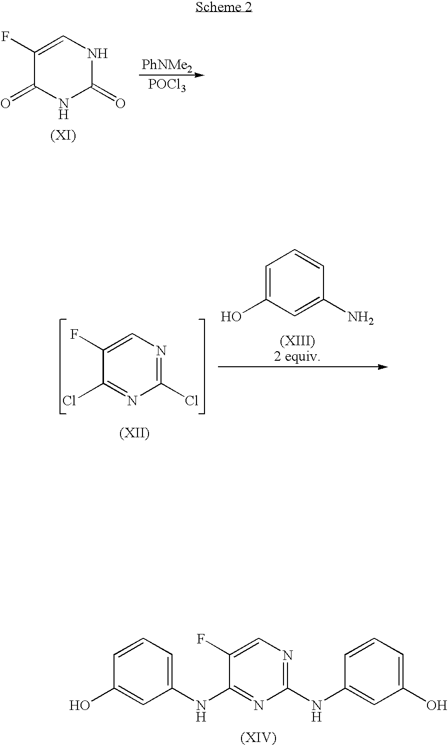

Although many of the synthetic schemes discussed above do not illustrate the use of protecting groups, skilled artisans will recognize that in some instances substituents R², R⁴, R⁵, R⁶, L¹ and/or L² may include functional groups requiring protection. The exact identity of the protecting group used will depend upon, among other things, the identity of the functional group being protected and the reaction conditions used in the particular synthetic scheme, and will be apparent to those of skill in the art. Guidance for selecting protecting groups and chemistries for their attachment and removal suitable for a particular application can be found, for example, in Greene & Wuts, supra.

Prodrugs according to structural formula (ii) may be prepared by routine modification of the above-described methods. Alternatively, such prodrugs may be prepared by reacting a suitably protected 2,4-pyrimidinediamine of structural formula (I) with a suitable progroup. Conditions for carrying out such reactions and for deprotecting the product to yield a prodrug of formula (ii) are well-known.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

All literature and patent references cited throughout the application are incorporated by reference into the application for all purposes.

What is claimed is:

1. A method of synthesizing a 2,4-pyrimidinediamine compound according to structural formula (I): 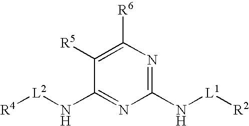
and salts, hydrates, solvates, N-oxides and prodrugs thereof, wherein:
L¹ and L² are each, independently of one another, selected from the group consisting of a direct bond and a linker;
R² is selected from the group consisting of (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;
R⁴ is selected from the group consisting of hydrogen, (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;
R⁵ is selected from the group consisting of R⁶, (C1-C6) alkyl optionally substituted with one or more of the same or different R⁸ groups, (C1-C4) alkanyl optionally substituted with one or more of the same or different R⁸ groups, (C2-C4) alkenyl optionally substituted with one or more of the same or different R⁸ groups and (C2-C4) alkynyl optionally substituted with one or more of the same or different R⁸ groups;
each R⁶ is independently selected from the group consisting of hydrogen, an electronegative group, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CF₃, —CH₂CF₃, —CF₂CF₃, —CN, —NC, —OCN, —SCN, —NO, —NO₂, —N₃, —S(O)R^{d}, —S(O)₂R^{d}, —S(O)₂OR^{d}, —S(O)NR^{c}R^{c}; —S(O)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙNR^{c}R^{c} and —[NHC(NH)]ₙNR^{c}R^{c}, (C5-C10) aryl optionally substituted with one or more of the same or different R⁸ groups, phenyl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups and 6-16 membered heteroarylalkyl optionally substituted with one or more of the same or different R⁸ groups;
R⁸ is selected from the group consisting of R^{a}, R^{b}, R^{a} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})—R^{b}, —NH—(CH₂)ₘ—R^{b}, —NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R^{a} is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b} is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, OC(O)NR^{c}R^{c}, OC(NH)NR^{c}R^{c}, OC(NR^{a})NR^{c}R^{c}, [NHC(O)]ₙR^{d}, —[NR^{a}C(O)]ₙR^{d}, —[NHC(O)]ₙOR^{d}, [NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c} is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each R^{d} is independently a protecting group or R^{a};
each m is independently an integer from 1 to 3; and
each n is independently an integer from 0 to 3,
comprising the steps of:
(a) treating a compound according to structural formula (II) with a phosphorous oxyhalide in an N,N-dialkylaniline at an elevated temperature 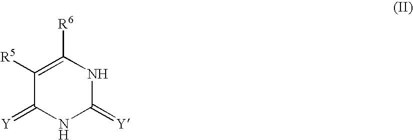
wherein Y and Y′ are each, independently of one another, selected from the group consisting of O and S, thereby forming a compound according to structural formula (III) 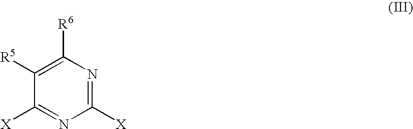
wherein each X is a halogen;
(b) treating compound (III) in a solvent at an elevated temperature with one equivalent of a compound according to structural formula (IV)
R⁴-L²-NH₂ (IV)
thereby forming a compound according to structural formula (V) 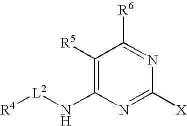
(V); and
(c) treating compound (V) in a solvent at an elevated temperature with one equivalent of a compound according to structural formula (VI)
R²-L¹-NH₂ (VI)
thereby forming compound (I), wherein R², R⁴, R⁵, R⁶, L¹ and L² are as defined above.

2. The method of claim 1 in which the temperature of step (b) is between about 80° C. to about 85° C.

3. The method of claim 1 in which the temperature of step (c) is between about 80° C. to about 85° C.

4. The method of claim 1 in which the phosphorous oxyhalide is phosphorous oxychloride.

5. The method of claim 1 in which the N,N-dialkylaniline is selected from the group consisting of N,N-diethylaniline and N,N-dimethylaniline.

6. The method of claim 1 in which the solvent in step (c) is isopropanol.

7. The method of claim 1 in which both (IV) and (VI) are 3-aminophenol.

8. The method of claim 1 in which in step (a) the phosphorous oxyhalide and N,N-dialkylaniline are refluxed.

9. The method of claim 8 in which the reaction product of step (a) is dissolved in a solvent and is further treated with an acid and water.

10. The method of claim 9 in which the solvent is dichloromethane.

11. The method of claim 9 in which the acid is hydrochloric acid.

12. The method of claim 10 further comprising the step of exchanging the dichloromethane with isopropanol.

13. The method of claim 1 in which in step (c) the solvent is isopropanol.

14. The method of claim 1 in which R⁵ of compound (II) is F.

15. The method of claim 14 in which Y and Y′ are both O.

16. The method of claim 15 in which R² and R⁴ are each 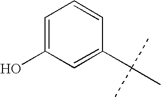
and L¹ and L² are each direct bonds.
